(19) United States
(12) Patent Application Publication
(10) Pub. No.: US 2005/0106116 A1
(43) Pub. Date: May 19, 2005

(54) Foot treatment method and composition

(76) Inventor: Donald L. Smothers, Terrell, TX (US)

Correspondence Address: TOD R NISSLE, PO BOX 55630, PHOENIX, AZ, 85078, US

(21) Appl. No.: 10/717,389

(22) Filed: Nov. 19, 2003

(51) Int. Cl. A61K 7/06

(52) U.S. Cl. 424/70.1

(57) ABSTRACT

A foot treatment composition includes water, an exfoliating composition, a bacteriacidal composition, a blood flow stimulating composition, and, a vasodialator composition.

This invention pertains to compositions and methods for treating the feet of an individual.

More particularly, the invention pertains to a method and composition for increasing blood circulation in the feet and for reducing the risk that infection or disease will arise in the feet.

Tiny blood vessels carry oxygen and nutrients to all areas of the body. When degradation of such blood vessels occurs, the extremities of the body—in particular the feet—are the first parts of the body to be affected. If capillaries in the feet fail to deliver oxygen and nutrients, nerve damage and gradual loss of feeling can occur. In addition, when circulation near the surface of the feet decreases, the skin can become dry, and lose flexibility and elasticity. This can lead to cracking of the skin and to ulcerations, infections, and—particularly in the case of patients who have diabetes—eventual amputation of the feet.

Accordingly, it would be highly desirable to provide a foot treatment method and composition that promotes the flow of blood in the feet and other extremities of an individual.

I have discovered a foot treatment composition and method that functions simultaneously to reduce the risk of infection in the feet, that removes dead skin to facilitate the growth and function of living skin cells, that stimulates blood flow, and that improves the ability of blood vessel to deliver oxygen and nutrients to the dermis and other tissue of the feet.

The new foot treatment method includes a first composition that includes natural components that form an exfoliant film, natural components that adhere to dead skin cells, natural antibacterial components, natural vasodilator components, and natural blood flow stimulation components. Natural components are components that occur in nature and that can be used as is or that can be extracted in their naturally occurring form and then used.

The natural exfoliant film forming components can comprise any desired components but presently are selected from a group consisting of polyvinyl alcohol, glycerin, mannan, and a thickener. The thicker can, by way of example and not limitation, include guar gum, carageenan, corn starch, zanthan gum, seaweed extract, or any other desired composition(s). Gums provide elasticity when incorporated in the composition of the invention. Any other desired components can be utilized to form a film when the composition is applied to the dermis of an individual. However, it is preferred that the film—after drying for a selected period of time—become sticky or tacky and, when rubbed with the individual's hand, disintegrate into small pieces that can be readily removed by washing with water or soap and water. Polyvinyl alcohol, mannan, and guar gum—for example—form such a film. The film functions to bind to dead cells on the outer surface of an individual's dermis and, when the film is rubbed, to roll or ball up and to remove or detach dead cells from the outer surface of the dermis. The friction generated when the film is rubbed off also facilitates penetration of the living dermis cells by the ginkgo biloba leaf extract and the chamomile flowers extract. Further the admixing of aloe barbadensis leaf liquid with ginkgo biloba leaf extract and chamomile flowers extract appears to facilitate penetration of the dermis by the ginkgo biloba leaf extract and chamomile flowers extract.

The natural antibacterial components can consist of any natural compositions that have antibacterial properties, but are presently selected from a group consisting of citrus aurantium and dulcis seed extract.

The natural blood flow stimulation components can consist of any natural components that stimulate the flow of blood in the blood vessel in a foot or other body extremity. However, the blood flow stimulation components are presently selected form a group consisting of chamomile flower extract and sage leaf extract. The blood flow stimulation components function to increase the rate of blood flow through at least surface blood vessels in the foot generally without dilating the blood vessels.

The vasodilator components can consist of any natural components that function to dilate the blood vessels in a foot or other extremity of the body. The preferred vasodilator is ginkgo biloba leaf extract. The combination of a vasodilator component with blood flow stimulation components is believed to be particularly useful in the practice of the invention in promoting blood flow in a patient's foot. It is important in the practice of the invention that the blood flow stimulation components and the vasocilator components be readily absorbed into and through the skin of a patient's foot.

The new foot treatment method can includes a second composition that is applied after the first composition and that functions to moisturize the skin, to improve the skin's natural flexibility and elasticity, and/or to increase blood flow. By way of example, and not limitation, the second composition can include glycerin, aloe barbadensis leaf, sorbitol, niacin, pantothenic acid, and cellulose.

The first composition presently includes from 30% to 95% by weight water, from 0.01% to 20%, preferably 1% to 15%, by weight aloe aloe barbadensis leaf or extract thereof, from 0.1% to 5.0% by weight of carbomer, from 1% to 20% by weight polyvinyl alcohol, from 1% to 10% by weight glycerin, from 0.1% to 5.0% by weight mannan, from 0.05% to 5.0% by weight guar gum, from 0.01% to 4.0%, preferably 0.5 to 4.0% by weight citrus aurantum or extract thereof, from 0.05% to 4.0% by weight pantothenic acid, from 0.01% to 15%, preferably 0.5% to 10%, by weight of ginkgo biloba leaf or extract thereof, from 0.01% to 15%, preferably 0.5% to 15%, by weight of chamomile flowers or leaf or extract thereof, and from 0.01% to 15%, preferably 0.5% to 10%, by weight of sage leaf or extract thereof. Carbomer is an acrylic acid based thickener.

The ginkgo bilboa leaf extract, chamomile flowers extract, and sage leaf extract are presently prepared by extracting into warmed aloe vera components from the powdered herb. These extracts are not prepared by extracting components from the powdered herb into water or alcohol or other liquids. The concentration of ginkgo bilboa leaf components and/or chamomile flowers components in the extract can vary widely and is typically in the range of 0.001% to 5.00%, preferably 0.5% to 5.00% by weight. The concentration of aloe vera components in the aloe barbadensis leaf can vary as desired, but typically is in the range of 1% to 100% by weight. One process for producing aloe barbadensis leaf liquid is simply to squeeze the liquid from aloe barbadensis leaves.

The aloe barbadensis leaf, polyvinyl alcohol, and glycerin function to adhere to dead skin cells. The mannan functions as a binder. The citrus aurantium, ginkgo biloba leaf extract, chamomile flowers extract also functions as exfoliants. The carbomer is a binder. Carrageenin or corn starch can also be used as binders or thickeners.

The second composition presently includes from 50% to 95% by weight glycerin, 0.01% to 15%, preferably 0.5% to 15% by weight aloe barbadensis leaf or extract thereof, 0.05% to 15% by weight sorbital, 0.01% to 10% pantothenic acid, 0.01% to 8% by weight niacin, and 0.01% to 4.0% by weight cellulose. Niacin and pantothenic acid function as circulatory stimulants. The glycerin and aloe barbadensis also function as emollients. Sorbitol is a moisturizer. Cellulose is a binder.

The following examples are provided by way of illustration, and not limitation, of the invention.

### EXAMPLE I

The following components are provided.

| Component | Weight % |
|---|---|
| Distilled water | 70.2 |
| Aloe Barbadensis leaf liquid [1] | 6.0 |
| Polyvinyl alcohol | 6.0 |
| Glycerin | 4.0 |
| Carbomer | 3.5 |
| Mannan | 0.5 |
| Guar | 0.5 |
| Citrus Aurantium extract [2] | 0.2 |
| Pantothenic acid | 0.1 |
| Ginkgo biloba leaf extract [3] | 4.0 |
| Chamomile flowers extract [4] | 4.0 |
| Sage leaf [5] | 4.0 |

[1] Nature's Way (TM) Aloe Vera Whole Leaf Juice, 99.7% Pure Certified Organic Aloe Vera. Ingredient includes whole leaf aloe vera juice, citric acid, Aloe PolyMax (TM) polysaccharide extract, potassium sorbate less than 0.1% and sodium benzoate less than 0.1% as preservatives.
[2] Nature's Way (TM) Bitter Orange Extract, Citrus Aurantium Standardized to 6% Synephrine. Ingredients include bitter orange dried extract, cellulose, maltodextrin, modified celullose gum, and silica.
[3] Nature's Way (TM) Gingko Biloba Extract, standardized to 24% Gingko flavone glycosides amd 6% terpene lactones. Other ingredients include gelatin and millet.
[4] Nature's Way (TM) Chamomile Extract standardized to 1.2% apigenin. Other ingredient is gelatin.
[5] Ground sage leaves.

The foregoing components are admixed as follows at room temperature to produce a first foot treatment composition.

1. The glycerin, mannan, and guar are mixed together to form a first homogeneous mixing composition.

2. The pantothenic acid is mixed in the distilled water until completely dissolved, after which the aloe barbadensis leaf fluid, citrus aurantium extract, ginkgo biloba leaf extract, and chamomile flowers extract are added to the water and mixed therein to form a second homogenous mixing composition.

3. The first mixing composition is mixed with the second mixing composition to form a homogenous third mixing composition.

4. The carbomer is added to the third mixing composition until a smooth homogeneous fourth mixing composition is produced.

5. The pH of the fourth mixing composition is adjusted to 6 with an alkaline agent like an amine, sodium hydroxide, potassium hydroxide to produce a fifth mixing composition.

6. The polyvinyl alcohol is admixed with the sixth mixing composition to produce the seventh, and final, mixing composition. The seventh mixing composition is a clear, translucent gel. The first foot treatment composition consists of the seventh mixing composition.

### EXAMPLE II

The following components are provided.

| Component | Weight % |
|---|---|
| Glycerin | 90.0 |
| Aloe barbadensis leaf liquid [1] | 5.0 |
| Sorbitol | 4.0 |
| Pantothenic acid | 0.2 |
| Niacin | 1.0 |
| Cellulose | 0.3 |

[1] Nature's Way (TM) Aloe Vera Whole Lead Juice, 99.7% Pure Certified Organic Aloe Vera. Ingredient includes whole leaf aloe vera juice, citric acid, Aloe PolyMax (TM) polysaccharide extract, potassium sorbate less than o.1% and sodium benzoate less than 0.1% as preservatives.

The foregoing components are admixed as follows at room temperature to produce a second foot treatment composition.

1. The glycerin and cellulose are admixed to produce a first mixing composition.

2. The sorbitol, pantothenic acid and niacin are mixed in the aloe barbadensis leaf liquid until dissolved to form a second mixing composition.

3. The first mixing composition and second mixing composition are admixed until homogenous to produce a clear translucent gel.

### EXAMPLE III

One teaspoon of the first foot treatment composition of Example I is applied to the patient's right foot. The composition can also, if desired, be applied to any other area of the user's body; however, the present primary application of the composition of Example I (and Example II) is for extremities like the feet, lower legs, hands, or forearms.

The first foot treatment composition is allowed to dry on the right foot until the composition becomes moderately sticky or tacky. The patient then utilizes the fingers of a hand to gently rub the composition to loosen the composition along with dead skin cells that adhere to the composition. The patient removes the composition by washing his right foot with a wash cloth and water.

After the patient finishes washing his right foot, he applies to the foot four to five drops of the second foot treatment composition of Example II and gently rubs with his hands the composition over the foot to form a thin film or layer. The user continues massaging the composition until the composition is completely, or nearly completely, absorbed into the skin. Any excess product is wiped off with a damp or dry wash cloth.

The foregoing procedure is repeated as desired. It is presently preferred, however, that the foregoing procedure be repeated twice daily for two weeks and thereafter once daily.

### EXAMPLE IV

Example I is repeated, except that the aloe barbadensis leaf liquid is omitted and an additional 6.0% by weight of distilled water is used instead. Similar results are obtained.

### EXAMPLE V

Example I is repeated, except that the ginkgo biloba leaf extract and chamomile flowers extract are omitted and an additional 8.0% by weight distilled water is used instead. Similar results are obtained.

### EXAMPLE VI

Ten adults between the ages of twenty and fifty are selected.

One teaspoon of the first foot treatment composition of Example I is rubbed over the entire surface of right foot of each adult.

One teaspoon of the foot treatment composition of Example IV is rubbed over the entire surface of the left foot of each adult.

The foot treatment compositions on the left and right feet of each adult are allowed to dry until the compositions becomes moderately sticky or tacky. Each adult then utilizes the fingers of a hand to gently rub the composition to loosen the composition along with dead skin cells that adhere to the composition. Each adult then completely removes the composition by washing his right foot with a wash cloth and water.

Each adult notes that the blood circulation in the right foot feels better than the blood circulation in the left foot. The color of the right foot of each adult is slightly pinker than the color of the left foot of each adult.

### EXAMPLE VII

Example VI is repeated except that the foot treatment composition of Example V is used in place of the foot treatment composition of Example IV. Similar results are obtained.

### EXAMPLE VII

Examples I and VI are repeated except that in Example I generic aloe vera whole leaf juice is utilized in place of the Nature's Way aloe vera. The generic aloe vera juice is 99% pure aloe vera. Similar results are obtained in Examples I and VI.

### EXAMPLE VIII

Examples I and VI are repeated except that in Example I (a) generic aloe vera whole leaf juice is utilized in place of the Nature's Way aloe vera juice, (b) 1% by weight of American Health and Herb™ 100% Organic sage tincture (Ingredients: sage, R/O water, 12% alcohol) is used in place of the 4% by weight sage leaves (c) 1% by weight of American Health and Herb Ginkgo Leaf Tincture (Ingredients: Ginkgo Leaf, R/O water, 12% alcohol) is used in place of the 4% by weight Nature's Way Ginkgo Biloba Extract, and (d) 1% by weight of American Health and Herb™ Chamomile Tincture (Ingredients: Chamomile 1 & 4, R/O water, 12% alcohol) is used in placed of the 4% by weight Nature's Way Chamomile Extract. The generic aloe vera juice is 99% pure aloe vera. Similar results are obtained in Examples I and VI.

### EXAMPLE IX

Examples I and VI are repeated except that in Example I (a) generic aloe vera whole leaf juice is utilized in place of the Nature's Way aloe vera juice, (b) 0.5% by weight of Now Foods™ 100% pure clary sage oil is used in place of the 4% by weight sage leaves (c) 0.1% by weight of Now Foods™ Neroli Oil w/7.5% Grape Seed Oil is used in place of the Nature's Way Bitter Orange Extract, and (d) 1% by weight Now Foods™ 1.0% by weight of 100% pure Chamomile oil. The generic aloe vera juice is 99% pure aloe vera. Similar results are obtained in Examples I and VI. The Nature's Way and Now Foods products noted above can be currently found on the store.yahoo.com web site.

Having described my invention in such terms as to enable those of skill in the art to make and practice it, and having described the presently preferred embodiments thereof,

1. A cleansing, antibacterial, blood flow stimulating, vasodialating, exfoliating foot treatment composition including
(a) 30% to 95% by weight water;
(b) a film forming composition;
(c) an exfoliating composition;
(d) a bacteriacidal composition;
(e) a blood flow stimulating composition; and,
(f) a vasodialating composition.

2. A foot treatment method comprising the steps of
(a) providing a foot treatment composition including
(i) 30% to 95% by weight water;
(ii) an exfoliating composition;
(iii) a bacteriacidal composition;
(iv) a blood flow stimulating composition;
(v) a vasodialating composition, and
(vi) a film forming composition;
(b) applying a layer of said foot treament composition to a patient's foot;
(c) allowing said layer of said composition to dry for a selected period of time; and,
(d) rubbing said dried layer to remove dead skin cells.
